(19) United States
(12) Patent Application Publication
(10) Pub. No.: US 2018/0255250 A1
(43) Pub. Date: Sep. 6, 2018

(54) PULSED, GATED INFRARED ILLUMINATED CAMERA SYSTEMS AND PROCESSES FOR EYE TRACKING IN HIGH AMBIENT LIGHT ENVIRONMENTS

(71) Applicant: Microsoft Technology Licensing, LLC, Redmond, WA (US)

(72) Inventors: Raymond Kirk Price, Redmond, WA (US); Michael Bleyer, Seattle, WA (US); Denis Demandolx, Bellevue, WA (US)

(21) Appl. No.: 15/449,189

(22) Filed: Mar. 3, 2017

(51) Int. Cl. H04N 5/33; A61B 3/113; G06T 7/70; G06T 7/20; H04N 5/225; G06T 7/60

(52) U.S. Cl. CPC H04N 5/33; A61B 3/113; G06T 7/70; G06T 2207/30196; H04N 5/2256; G06T 7/60; G06T 7/20

(57) ABSTRACT

An eye movement tracking device includes an illumination source configured to transmit energy from a location proximate to an eye of a person such that a portion of transmitted energy is reflected off the eye of the person, a filter configured to generate filtered reflections, and an image sensor and shutter configured to detect the filtered reflections and to distinguish the filtered reflections of the portion of the transmitted energy from other energy detected by the image sensor and shutter based on times of flight and the frequency band of the filtered reflections of the portion of the transmitted energy and the other energy. The eye tracking device further includes a processor configured to determine a position of the eye of the person.

### BACKGROUND OF THE DISCLOSURE

Various applications utilizing eye tracking technology, also known as gaze tracking, are evolving and becoming an important part of next generation human-computer interfaces. Eye tracking technology has many potential applications including entertainment applications, research applications, interaction tool applications, such as for people who are physically impaired, virtual reality applications, augmented reality applications, military applications, and other similar applications.

Typical eye tracking systems use infrared (IR) cameras with IR light sources to detect the pupil/iris. Additionally, these systems generally use either direct imaging or indirect imaging. Direct imaging systems image the eye region directly by placing one or more IR sensors directly aimed at the eyes. Both of these types of imaging systems have interference problems with ambient light.

In this regard, ambient light is a significant issue for eye tracking systems in, for example, augmented reality systems. Even seemingly small amounts of ambient light can cause significant amounts of interference on the eye tracking systems, as ambient light impinging on optical surfaces, such as the Augmented Reality system protective optical surfaces, is reimaged by the camera sensor.

A typical prior art eye tracking approach uses low power LEDs operating for a long illumination time (˜2 milliseconds (ms)) and a global shutter silicon sensor operating with long exposure time (˜2 ms). This solution is subject to substantial interference with ambient light as described above. Moreover, the low power LEDs of the prior art eye tracking approach have an extensive far field illumination profile as illustrated in FIG. 13 that results in higher power usage. Additionally, the other components of the prior art eye tracking approach also have higher power usage due to long illumination time, long exposure time, and the like.

### SUMMARY OF THE DISCLOSURE

In one aspect, an eye movement tracking device includes an illumination source configured to transmit energy within a frequency band from a location proximate to an eye of a person such that a portion of transmitted energy is reflected off the eye of the person, a filter configured to filter a portion of the transmitted energy that is reflected off the eye of the person to generate filtered reflections, an image sensor and shutter configured to detect the filtered reflections of the portion of the transmitted energy, and to distinguish the filtered reflections of the portion of the transmitted energy from other energy detected by the image sensor and shutter based on times of flight and the frequency band of the filtered reflections of the portion of the transmitted energy and the other energy, and a processor configured to use the filtered reflections of the portion of the transmitted energy to determine a position of the eye of the person.

In another aspect, a process of tracking eye movement of a person includes transmitting energy from an illumination source within a frequency band from a location proximate to an eye of the person such that a portion of the transmitted energy is reflected off the eye of the person, filtering the portion of the transmitted energy that is reflected off the eye of the person to generate filtered reflections with a filter, detecting the filtered reflections of the portion of the transmitted energy with an image sensor and shutter, and distinguishing the filtered reflections of the portion of the transmitted energy from other energy detected by the image sensor and shutter based on times of flight and said frequency band of the filtered reflections of the portion of the transmitted energy and the other energy, and determining a position of the eye of the person based on the filtered reflections of the portion of the transmitted energy with a processor.

In another aspect, an eye movement tracking device includes means for transmitting energy within a frequency band from a location proximate to an eye of a person such that a portion of the transmitted energy is reflected off the eye of the person, means for filtering the portion of the transmitted energy that is reflected off the eye of the person to generate filtered reflections, means for detecting the filtered reflections of the portion of the transmitted energy, and means for distinguishing the filtered reflections of the portion of the transmitted energy from other energy detected based on times of flight and said frequency band of the filtered reflections of the portion of the transmitted energy and the other energy, and means for determining a position of the eye of the person based on the filtered reflections of the portion of the transmitted energy.

Additional features, advantages, and aspects of the disclosure may be set forth or apparent from consideration of the following detailed description, drawings, and claims. Moreover, it is to be understood that both the foregoing summary of the disclosure and the following detailed description are exemplary and intended to provide further explanation without limiting the scope of the disclosure as claimed.

### BRIEF DESCRIPTION OF THE DRAWINGS

The accompanying drawings, which are included to provide a further understanding of the disclosure, are incorporated in and constitute a part of this specification, illustrate aspects of the disclosure and together with the detailed description serve to explain the principles of the disclosure. No attempt is made to show structural details of the disclosure in more detail than may be necessary for a fundamental understanding of the disclosure and the various ways in which it may be practiced. In the drawings:

FIG. 1 illustrates a schematic of an eye tracking system according to at least one aspect of the disclosure.

FIG. 2 illustrates a back view of an eye tracking system according to at least one aspect of the disclosure.

FIG. 3 illustrates a perspective front view of the eye tracking system of FIG. 2 according to at least one aspect of the disclosure.

FIG. 4 is a block diagram further illustrating an aspect of the eye tracking system according to at least one aspect of the disclosure.

FIG. 5 illustrates a far field illumination profile according to aspects of the disclosure.

FIG. 6 illustrates illuminant spectral distribution at different temperatures according to aspects of the disclosure.

FIG. 7 illustrates a graph of the optical transmission of a filter according to an aspect of the disclosure.

FIG. 8 illustrates an exploded view of lenses and the filter along with a holder according to an aspect of the disclosure.

FIG. 9 illustrates a cross-sectional view of the lenses and the filter along with the holder according FIG. 8.

FIG. 10 illustrates a cross-sectional view of the lenses and the filter along with exemplary light transmission according to an aspect of the disclosure.

FIG. 11 graphically illustrates operation of the system using multiple short laser pulses in comparison to prior art systems.

FIG. 12 is a flow diagram showing an example of the operational process of the eye tracking system according to some aspects of the disclosure.

FIG. 13 illustrates a far field illumination profile of prior art LEDs.

### DETAILED DESCRIPTION OF THE DISCLOSURE

The aspects of the disclosure and the various features and advantageous details thereof are explained more fully with reference to the non-limiting aspects and examples that are described and/or illustrated in the accompanying drawings and detailed in the following description. It should be noted that the features illustrated in the drawings are not necessarily drawn to scale, and features of one aspect may be employed with other aspects as the skilled artisan would recognize, even if not explicitly stated herein. Descriptions of well-known components and processing techniques may be omitted so as to not unnecessarily obscure the aspects of the disclosure. The examples used herein are intended merely to facilitate an understanding of ways in which the disclosure may be practiced and to further enable those of skill in the art to practice the aspects of the disclosure. Accordingly, the examples and aspects herein should not be construed as limiting the scope of the disclosure, which is defined solely by the appended claims and applicable law. Moreover, it is noted that like reference numerals represent similar parts throughout the several views of the drawings.

In this description, references to “an aspect,” “one aspect” or the like, mean that the particular feature, function, structure or characteristic being described is included in at least one aspect of the technique introduced here. Occurrences of such phrases in this specification do not necessarily all refer to the same aspect. On the other hand, the aspects referred to also are not necessarily mutually exclusive.

In this disclosure multiple techniques for mitigating the effects of ambient light in eye tracking systems used in augmented reality systems, virtual reality systems, gaming systems, medical systems, military systems, engineering systems, and the like are addressed. Prior art solutions are extremely sensitive to ambient illumination. The disclosure provides a solution that isolates the returned light signal to specific distances. The advantages of this approach include at least: reduced sensitivity to ambient light, reduced overall illumination power, and reduced sensitivity to parasitic reflections/stray light from other IR emitting sensors.

The disclosure may also use a time gated sensor with a pulsed laser/LED to truncate light pulses to slices in depth from the light source. By using coordinated fast light pulses and fast electronic shutter pulses to the sensor, the response is limited to a specific distance that is defined as the time delay between the light pulse and the sensor. Moreover, the disclosed solution may utilize a narrow spectral linewidth light source, the light source being configured to provide extremely short pulses (sub nanoseconds (ns) to a few ns in pulse width), a fast (approximately same pulse length as the light source) global shutter sensor, and a timing mechanism that coordinates the pulses from the laser and the sensor shutter. The light source operating with the extremely short pulses may also be beneficial to the user as the user's eyes are subjected to less light from the light source. This reduces the amount of light the user's eyes are exposed to.

Additionally, the disclosure may also use a filter, such as an infrared bandpass filter, that may filter out or substantially filter out ambient light received by the sensor. In a particular aspect, the filter may be implemented as a narrow bandpass filter. Use of the narrow bandpass filter may include a number of advantages that may include further reduced sensitivity to ambient light and further reduced sensitivity to parasitic reflections.

Additionally, prior art solutions operated the LEDs in quasi continuous wave (CW) mode, with no pulse power enhancement. In some aspects, the disclosed system may operate by using multiple short laser or LED pulses and coordinating the sensor shutter with the light pulses. In this regard, the total integration time can be reduced by the power enhancement achieved with laser or LED light pulses. A typical value achievable using this technique is 2-10× the CW output power value.

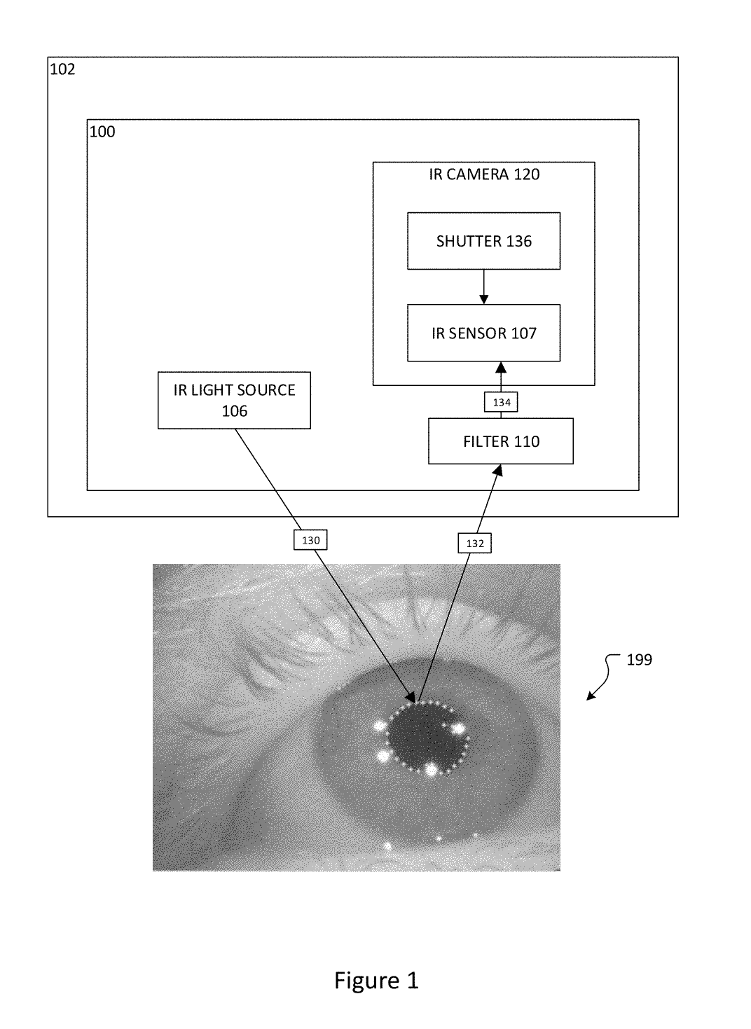

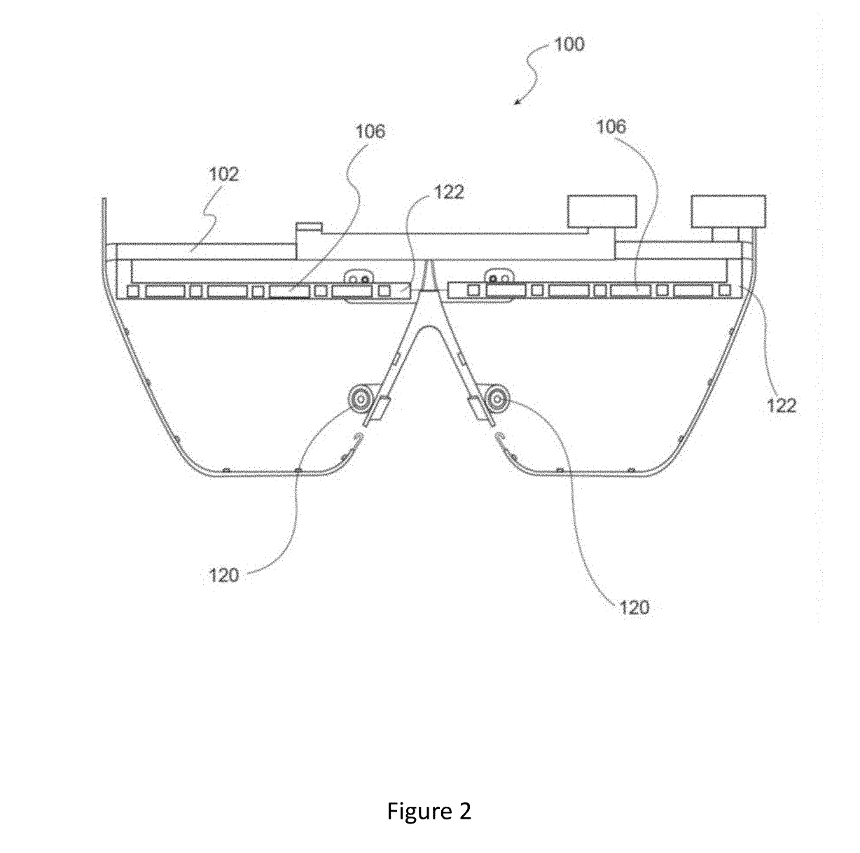

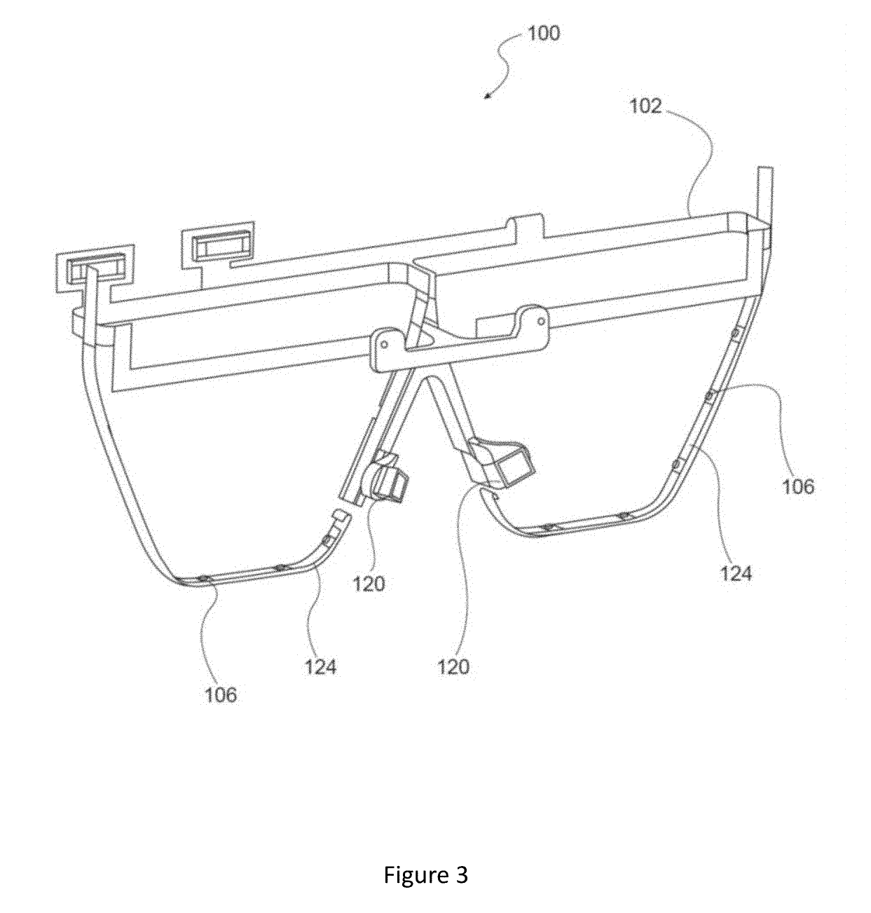

FIG. 1 illustrates a schematic of an eye tracking system according to at least one aspect of the disclosure; FIG. 2 illustrates a back view of an eye tracking system according to at least one aspect of the disclosure; and FIG. 3 illustrates a perspective front view of the eye tracking system of FIG. 2 according to at least one aspect of the disclosure. As shown, the eye tracking system 100 may be mounted to support frames 102 proximate to at least one of the user's eyes. “Proximate” in this context means within a few centimeters, such as: less than 1 cm, less than 2 cm, less than 3 cm, less than 4 cm, or less than 5 cm. What constitutes proximate may be dependent on the shape and configuration of the support frames 102, the physiology of the user including facial structure, and the like.

Note that the physical shape of the support frames 102 as shown in FIG. 2 and FIG. 3 is just one of many possible examples of the shape the support frames 102 may have. In some aspects, the eye tracking system 100 may not include the support frames 102 and may be implemented with a fastener (e.g., a spring-loaded clip, Velcro, or the like) to detachably connect the eye tracking system 100 to eyeglass frames. The fastener can be a “universal” fastener capable of mounting the device to any standard eyeglasses, or it can be designed specifically for a given model or manufacturers eyeglasses.

In some aspects, the eye tracking system 100 may not include the support frames 102 and may be implemented in another device such as a personal computer, a laptop, a workstation, and the like and may operate otherwise consistent with the disclosure except operation at greater distances, such as 30 cm-90 cm.

In some aspects, the support frames 102 may be wearable. In some aspects, the support frames 102 may be configured to operate in conjunction with a headset for gaming systems, augmented reality systems, virtual reality systems, medical systems, military systems, engineering systems, and/or like systems. In some aspects, the support frames 102 may be configured to operate in conjunction with one or more of a head-mounted display, a helmet-mounted display (for users that are utilizing a helmet such as for aviation applications), an optical head-mounted display, or the like. In some aspects, the support frames 102 may be wearable with a configuration to implement augmented reality applications, virtual reality applications, gaming applications, medical applications, military applications, engineering applications, and the like.

As discussed further below, the eye tracking system 100 has at least one IR light source 106, a filter 110, and an IR camera 120 that includes an IR sensor 107. In one aspect, the IR camera 120 may be implemented as a gated “fast global shutter” IR camera.

In this regard, the IR camera 120 may include an electronic shutter 136 for gating the IR sensor 107 on or off, which is controllable to selectively have low or high transmittance. The shutter 136 is said to be “closed” when it is not collecting photons for the frame exposure and gates the IR sensor 107 off, and is said to be “open” when it is collecting photons for the frame exposure and gates the IR sensor 107 on. A “gate” refers to a period during which IR sensor 107 is gated on by the shutter 136 and the IR sensor 107 integrates light for the frame exposure. In some aspects, the shutter 136 is implemented as a global shutter to globally shutter the IR sensor 107. A controller controls pulsing of the IR light source 106 and operation of the shutter 136 to gate the IR sensor 107. In one aspect, the controller controls and applies a delay between an operation of the electronic shutter 136 for gating the IR sensor 107 to receive a light pulse from the IR light source 106 by operation of the shutter 136 such that opening of the shutter 136 is coordinated to account for the round trip time between the light pulse illuminators and the eye 199. In other words, the controller controls an operation of the electronic shutter 136 for the time-of-flight of a light pulse such that opening of the shutter 136 is coordinated to account for the time-of-flight to and from the eye. In one aspect, the controller is implemented as a CPU 422 described in further detail below. In another aspect, the control signaling for the IR sensor 107, the shutter 136, and/or the IR light source 106, such as LEDs, may come from an off-board Application Specific Integrated Circuit. In another aspect, the control signal for the IR sensor 107, the shutter 136, and/or the IR light source 106, such as LEDs, may come from a control circuitry logic and precision timing control circuitry on an image sensor or the IR sensor 107.

In one aspect, the IR light source 106 may be implemented as a single light source. In another aspect, the IR light source 106 may be implemented as a plurality light sources. In one aspect, the IR light source 106 may be implemented as a plurality light sources for each eye. In one aspect, the IR light source 106 may be implemented as a plurality light sources arranged along a bridge portion 122 of the support frames 102 as shown in FIG. 2. In one aspect, the IR light source 106 may be implemented as a plurality light sources arranged along a lower frame portion 124 of the support frames 102 as shown in FIG. 3. In one aspect, the IR light source 106 may be implemented as a plurality light sources arranged along a bridge portion 122 of the support frames 102 as shown in FIG. 2 and may be further implemented as a plurality light sources arranged along a lower frame portion 124 of the support frames 102 as shown in FIG. 3. For brevity, the IR light source 106 whether implemented singularly or as a plurality will be referred to simply as the IR light source 106 throughout the disclosure.

In one aspect, the IR camera 120 may be implemented as a single camera. In another aspect, the IR camera 120 may be implemented as a plurality cameras. In a particular aspect, the IR camera 120 may be implemented as two cameras, one for each eye, arranged on the lower frame portion 124. In a particular aspect, the IR camera 120 may be implemented as a plurality cameras arranged on the lower frame portion 124. For brevity, the IR camera 120 whether implemented singularly or as a plurality will be referred to simply as the IR camera 120 throughout the disclosure.

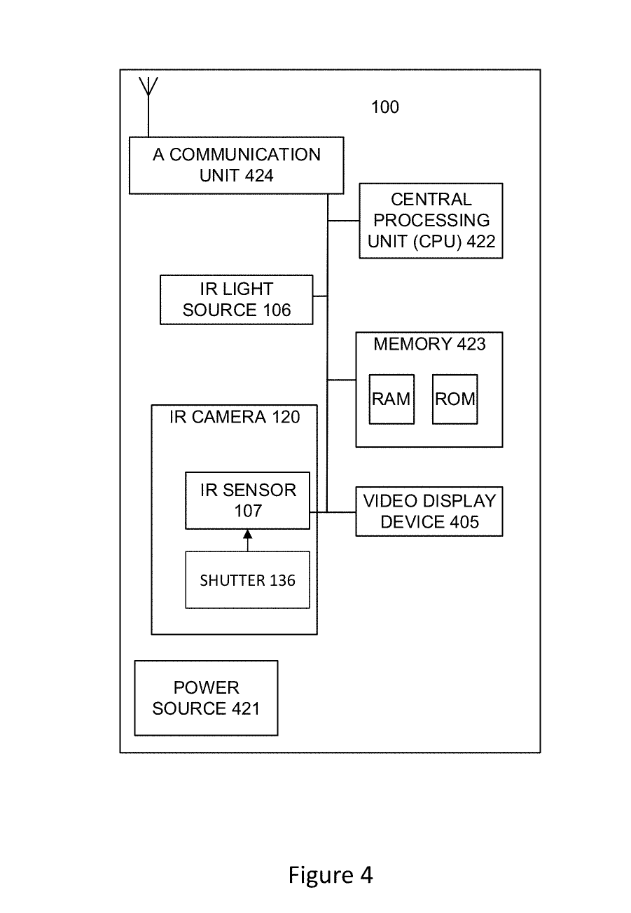

FIG. 4 is a block diagram further illustrating an aspect of the eye tracking system according to at least one aspect of the disclosure. As illustrated, the eye tracking system 100 may include a power source 421 (e.g., a battery), a central processing unit (CPU) 422, a memory 423, the IR camera 120 including the IR sensor 107 and shutter 136, the IR light source 106, a human-visible spectrum video display device 405, and a communication unit 424. The IR camera 120 may further include lenses associated with the IR camera 120 as well as the filter 110 as described in further detail below.

The video display device 405 can be any conventional video display device. In some aspects, the video display device 405 may generate a RGB video image. The RGB video image generated by the video display device 405 may be associated with a virtual reality application, an augmented reality application, a military application, gaming application, medical application, engineering application, or the like application. In some aspects of the disclosure the video display device 405 may not be utilized. In some aspects, the video display device 405 may be implemented as cathode ray tubes (CRT), liquid crystal displays (LCDs), liquid crystal on silicon (LCoS), organic light-emitting diodes (OLED), or the like display technology.

The IR camera 120 can be a fast shutter gated time-of-flight (TOF) IR camera. Note that in some aspects, the IR camera 120 may include its own processor and/or memory (not shown), separate from the CPU 422 and memory 423, for performing image capture and/or image processing operations.

In some aspects, the CPU 422 controls operation of the other components of the eye tracking system 100 and determines gaze direction or performs eye tracking computations related to gaze determinations. In some aspects, the CPU 422 controls pulsing of the IR light source 106 and operation of the shutter 136 to gate the IR sensor 107. The CPU 422 may be or may include any known or convenient form of processor and/or controller, such as an appropriately programmed general-purpose microprocessor, special-purpose microprocessor, digital signal processor, programmable microcontroller, application-specific integrated circuit (ASIC), programmable logic device (PLD), or the like, or a combination of any two or more such devices. Further, if the CPU 422 is a programmable microprocessor, it can be either a single-core processor or a multicore processor. In some aspects, the CPU 422 may be a special processor, such as image capturing processor.

The memory 423 can be used to store any one or more of: the image data acquired by the IR camera 120, program code for execution by the CPU 422, intermediate data resulting from computations or calculations by the CPU 422, image data for the video display device 405 or other data and/or program code. Hence, portions of the memory 423 can actually reside in the CPU 422, the video display device 405, and/or the IR camera 120. The memory 423 can include one or more physical storage devices, which may be or may include random access memory (RAM), read-only memory (ROM) (which may be erasable and programmable), flash memory, miniature hard disk drive, or other suitable type of storage device, or a combination of such devices.

The communication unit 424 enables the eye tracking system 100 to communicate with an external device or system (not shown), such as a computer or other type of processing device. For example, in certain aspects, at least some of the eye tracking computations may be implemented by the external device (e.g., a personal computer), based on data acquired by the eye tracking system 100 and transmitted to the external device by the communication unit 424. This may allow the programming or configuration of the CPU 422 to be made much simpler, or it may allow the CPU 422 to be replaced by a much simpler type of controller, or even omitted entirely from the eye tracking system 100. The communication unit 424 can be or include a transceiver that performs wired communication, wireless communication, or both. For example, the communication unit 424 can be or include any one or more of: a universal serial bus (USB) adapter, Ethernet adapter, modem, Wi-Fi adapter, cellular transceiver, baseband processor, Bluetooth or Bluetooth Low Energy (BLE) transceiver, a device configured to operate on a communication channel as defined herein, or the like, or a combination thereof.

Each IR light source 106 of the eye tracking system 100 can be or may include, for example, one or more light emitting diodes (LEDs), laser sources, and/or the like. The IR light source 106 may be used in conjunction with TOF principles to provide high quality depth determination, such as for use in eye tracking, gesture recognition, object recognition, and the like. As discussed further below, the illumination by the IR light source 106 may be controlled such that for each shutter window of an imaging frame, the illumination can be set on or off. For aspects in which there is more than one IR light source 106, the eye tracking system 100 may be able to turn on or off each source independently.

FIG. 1 illustrates the principle of operation of the eye tracking device, according to at least one aspect. Note that FIG. 1 is intended to be schematic in nature, such that the actual positions of the IR light source 106 and the IR sensor 107 in an actual implementation may differ from their positions as shown in FIG. 1. The IR light source 106 transmits IR energy 130 toward the user's eye 199. A portion 132 of the transmitted IR energy may be reflected off the user's eye 199 back to the filter 110. The filter 110 filters the portion 132 and delivers a filtered IR energy 134 to the IR sensor 107 of the IR camera 120. The filtered IR energy 134 that reaches the IR sensor 107 is detected and used by the CPU 422 (or alternatively by an external device) to determine the eye position (i.e., using pupil and/or iris identification), eye tracking, gesture recognition, object recognition, and the like.

In one aspect, once the eye position is determined, it is possible to identify the gaze location, such as on a RGB video image by using standard methods for gaze tracking. One way to accomplish this is by using a polynomial to map a pupil center or a pupil-glint vector to the RGB coordinates. The gaze location may be used for a number of applications. For example, in some aspects that include high definition graphic rendering, gaze tracking or point of interest tracking may be utilized to modify a location of a display of the high definition graphics such that the image is rendered within the field of view of the user as determined by the eye tracking system 100. This reduces computational power required of the CPU 422 by limiting generation of the high definition graphics.

The IR light source 106 may be configured for mitigating the effects of ambient light in eye tracking systems used in augmented reality systems, virtual reality systems, gaming systems, medical systems, military systems, engineering systems, and the like systems. As noted above, previous solutions were extremely sensitive to ambient illumination. In this regard, the IR light source 106 may be configured for extremely short pulses (sub nanosecond (ns) to a few ns in pulse width). In particular, the IR light source 106 may be configured to emit short light pulses in the range of 0.01 ns to 70 ns; the IR light source 106 may be configured to emit short light pulses in the range of 0.01 ns to 0.1 ns; the IR light source 106 may be configured to emit short light pulses in the range of 0.1 ns to 1 ns; the IR light source 106 may be configured to emit short light pulses in the range of 1 ns to 10 ns; the IR light source 106 may be configured to emit short light pulses in the range of 10 ns to 20 ns; the IR light source 106 may be configured to emit short light pulses in the range of 20 ns to 30 ns; the IR light source 106 may be configured to emit short light pulses in the range of 30 ns to 40 ns; the IR light source 106 may be configured to emit short light pulses in the range of 40 ns to 50 ns; the IR light source 106 may be configured to emit short light pulses in the range of 50 ns to 60 ns; and/or the IR light source 106 may be configured to emit short light pulses in the range of 60 ns to 70 ns. In this regard, the IR light source 106 configured for extremely short pulses may achieve improved ambient performance. In further aspects, the IR light source 106 may be configured for extremely short repeated pulses to provide the total dose required by the imaging system.

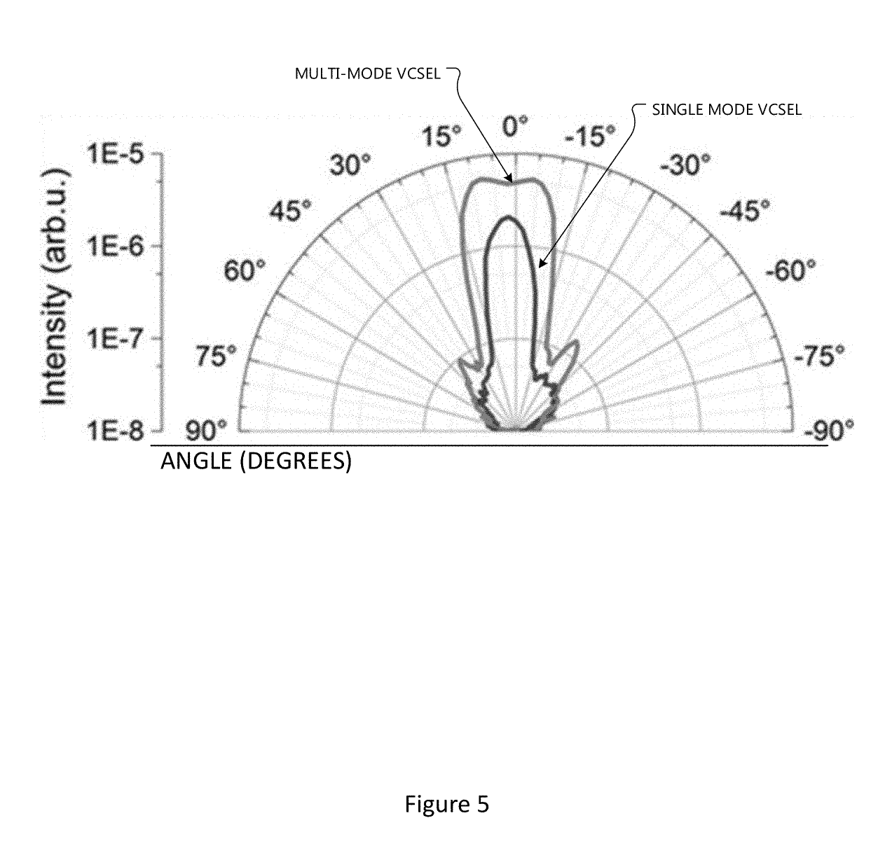

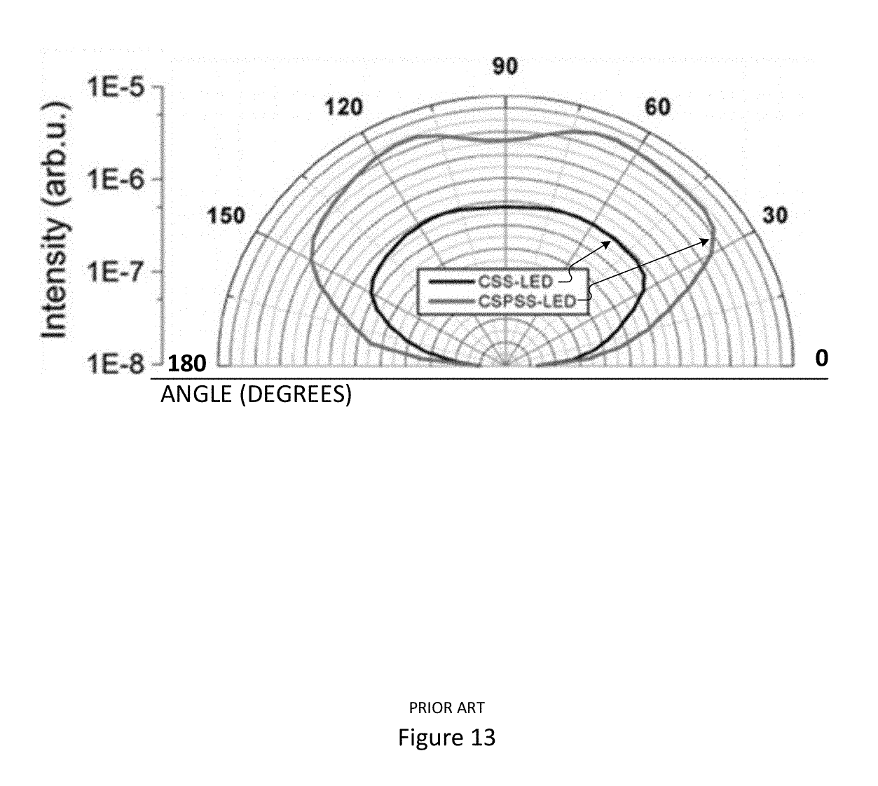

FIG. 5 illustrates a far field illumination profile according to aspects of the disclosure. In particular, FIG. 5 illustrates an intensity profile of a multimode Vertical Cavity Surface Emitting Laser (VCSEL) and a single mode Vertical Cavity Surface Emitting Laser (VCSEL) implementations of the IR light source 106. FIG. 5 uses arbitrary units for intensity along the vertical axis as indicated on the left side of the profile and further shows an angle in degrees along a horizontal axis. In this regard, it should be apparent that the FIG. 5 VCSEL results in a much narrower intensity profile in comparison to the LEDs implemented in the prior art that have a much wider intensity profile as illustrated in FIG. 13.

In this regard, the IR light source 106 of the disclosure may be configured with a low output divergence consistent with aspects of FIG. 5. However, other output divergences are contemplated as well. Typical LEDs have a divergence of approximately 100 degrees as illustrated in FIG. 13. Additionally, if an LED is to be used as the IR light source 106, a lens can be used to reduce the divergence angle of the LED light emission. In some aspects, the IR light source 106 may be implemented to have an output divergence less than 90°; the IR light source 106 may be implemented to have an output divergence less than 80°; the IR light source 106 may be implemented to have an output divergence less than 70°; the IR light source 106 may be implemented to have an output divergence less than 60°; the IR light source 106 may be implemented to have an output divergence less than 50°; the IR light source 106 may be implemented to have an output divergence less than 40°; the IR light source 106 may be implemented to have an output divergence less than 30°; and/or the IR light source 106 may be implemented to have an output divergence less than 20°. In this regard, the IR light source 106 that has an output divergence of less than 20° when compared to the prior art output divergence of 100° results in a ratio of (20/2)²/(100/2)², which indicates this aspect achieves a 25× reduction in the required illumination power for the application. Accordingly, the IR light source 106 may utilize less power than the prior art light sources.

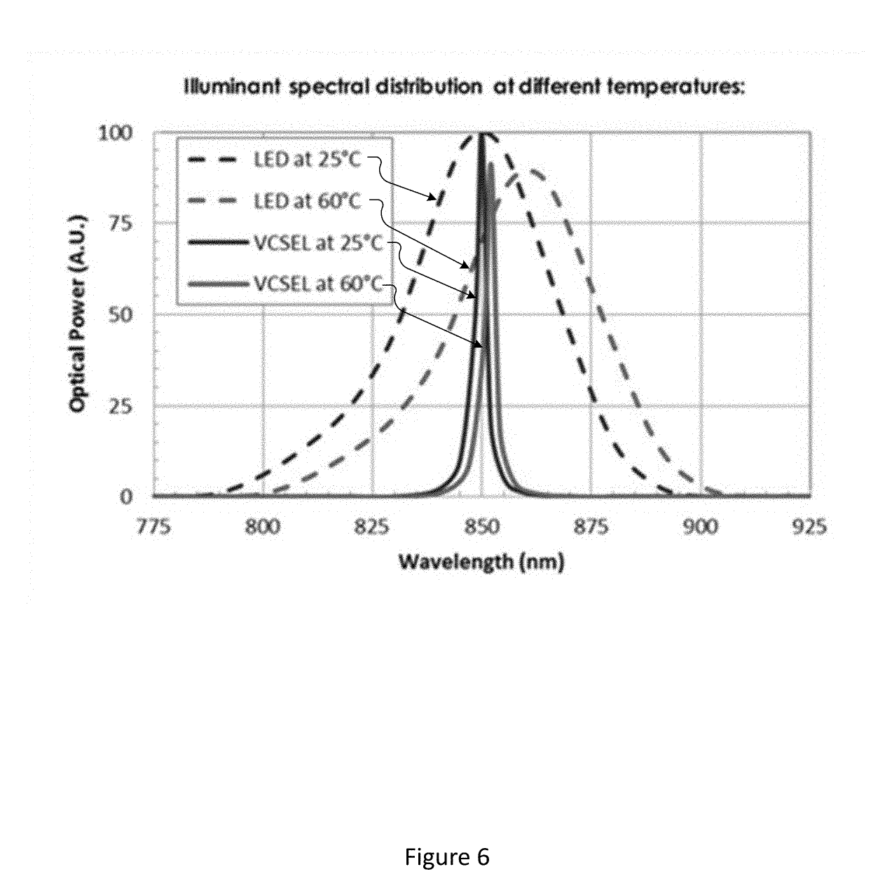

FIG. 6 illustrates illuminant spectral distribution at different temperatures according to aspects of the disclosure. FIG. 6 illustrates a graph of illuminant spectral distribution at different temperatures for two different devices, an LED and VCSEL-based illumination system, at 25 and 60 degrees C. FIG. 6 shows optical power utilizing arbitrary units along a vertical axis and spectral wavelength along a horizontal axis in nanometers (nm). Typical LEDs have a spectral linewidth of approximately 100 nanometers as illustrated in FIG. 6. In particular, FIG. 6 illustrates a LED operating at 25° C., and the same LED operating at 60° C., both having a spectral linewidth of approximately 100 nanometers. Moreover, FIG. 6 illustrates a Vertical Cavity Surface Emitting Laser (VCSEL) operating at 25° C. and 60° C. It should be clear from FIG. 6 that the VCSEL implementations of the IR light source 106 have a spectral linewidth much narrower than the LEDs.

In this regard, the IR light source 106 may be configured as a narrow spectral linewidth light source. The IR light source 106 according to the disclosure may have a spectral linewidth less than 80 nanometers (nm); the IR light source 106 according to the disclosure may have a spectral linewidth less than 70 nm; the IR light source 106 according to the disclosure may have a spectral linewidth less than 60 nm; the IR light source 106 according to the disclosure may have a spectral linewidth less than 50 nm; the IR light source 106 according to the disclosure may have a spectral linewidth less than 40 nm; the IR light source 106 according to the disclosure may have a spectral linewidth less than 30 nm; the IR light source 106 according to the disclosure may have a spectral linewidth less than 20 nm; the IR light source 106 according to the disclosure may have a spectral linewidth less than 10 nm; the IR light source 106 according to the disclosure may have a spectral linewidth less than 5 nm; and/or the IR light source 106 according to the disclosure may have a spectral linewidth less than 1 nm. In one aspect, the IR light source 106 may be defined based on a wavelength that is Full Width Half Max (FWHM). In one aspect, the IR light source 106 may be implemented utilizing LEDs that have a FWHM of approximately 30 nm. In one aspect, the IR light source 106 may be implemented utilizing Lasers that have a FWHM of 5 nm. In one aspect, the IR light source 106 may be implemented utilizing wavelength stabilized devices, like VCSELs, that have a FWHM of approximately 1 nm. In one aspect, the IR light source 106 may be implemented utilizing an LED light source or a laser light source. If a LED IR light source is used, then the bandpass filter may remove a substantial portion of the IR light. In one aspect, the IR light source 106 may be implemented utilizing a VCSEL laser that provides reduced spectral linewidth, and may be easily integrated with a narrow IR bandpass filter, resulting in a more efficient use of photons.

In one aspect, the IR light source 106 according to the disclosure may operate consistent with the spectral linewidth described above and centered on a range of 830 nm-870 nm. In one aspect, the IR light source 106 according to the disclosure may operate consistent with the spectral linewidth described above and centered on a range of 840 nm-860 nm. In one aspect, the IR light source 106 according to the disclosure may operate consistent with the spectral linewidth described above and centered on a range of 845 nm-855 nm. In one aspect, the IR light source 106 according to the disclosure may operate consistent with the spectral linewidth described above and centered on about 850 nm. Other aspects may have a wavelength of the illuminator and bandpass filter centered at 940 nm.

In one aspect, the light source may be implemented as a low divergence VCSEL with an output divergence within the above-noted ranges. In one aspect, the light source may be implemented as a VCSEL with an output divergence of about 20 degrees. In one aspect, the light source may be implemented as a VCSEL with a spectral linewidth within the above-noted ranges. In one aspect, the light source may be implemented as a VCSEL with spectral linewidth less than 5 nm. In one aspect, the light source may be implemented as a VCSEL configured to emit short light pulses within the above-noted ranges.

The shutter 136 may be configured for mitigating the effects of ambient light in eye tracking systems used in augmented reality systems, virtual reality systems, gaming systems, medical systems, military systems, engineering systems, and the like systems. As noted above, previous solutions were extremely sensitive to ambient illumination. In this regard, the shutter 136 may be configured for gating the IR light source 106 for extremely short pulses (sub nanosecond (ns) to a few ns in pulse width). In particular, the shutter 136 may be configured to gate the IR light source 106 for short light pulses in the range of 0.01 ns to 70 ns; the shutter 136 may be configured to gate the IR light source 106 for short light pulses in the range of 0.01 ns to 0.1 ns; the shutter 136 may be configured to gate the IR light source 106 for short light pulses in the range of 0.1 ns to 1 ns; the shutter 136 may be configured to gate the IR light source 106 for short light pulses in the range of 1 ns to 10 ns; the shutter 136 may be configured to gate the IR light source 106 for short light pulses in the range of 10 ns to 20 ns; the shutter 136 may be configured to gate the IR light source 106 for short light pulses in the range of 20 ns to 30 ns; the shutter 136 may be configured to gate the IR light source 106 for short light pulses in the range of 30 ns to 40 ns; the shutter 136 may be configured to gate the IR light source 106 for short light pulses in the range of 40 ns to 50 ns; the shutter 136 may be configured to gate the IR light source 106 for short light pulses in the range of 50 ns to 60 ns; and/or the shutter 136 may be configured to gate the IR light source 106 for short light pulses in the range of 60 ns to 70 ns. In this regard, the shutter 136 configured for extremely short pulses may achieve reduced power consumption for IR light source 106. Moreover, the electronic shutter 136 may gate the IR sensor 107 to receive a light pulse from the IR light source 106 by operation the shutter 136 such that opening of the shutter 136 is coordinated to account for the round trip time between the light pulse illuminators and the eye.

In one aspect, the light source may be implemented as a low divergence VCSEL with an output divergence within the above-noted ranges, an output bandwidth within the above-noted ranges, and configured to emit short light pulses within the above-noted ranges.

In some aspects, the filter 110 may be implemented as an IR bandpass filter. The bandpass implementation of the filter 110 may be configured to pass frequency ranges consistent with the bandwidth of the IR light source 106 and attenuate frequency ranges outside the bandwidth of the IR light source 106. In this regard, any ambient light outside the narrow bandpass of the filter 110 will be attenuated. Accordingly, the IR sensor 107 may subsequently have reduced sensitivity to ambient light, reduced sensitivity to parasitic reflections/stray light from other IR emitting sensors, and the like.

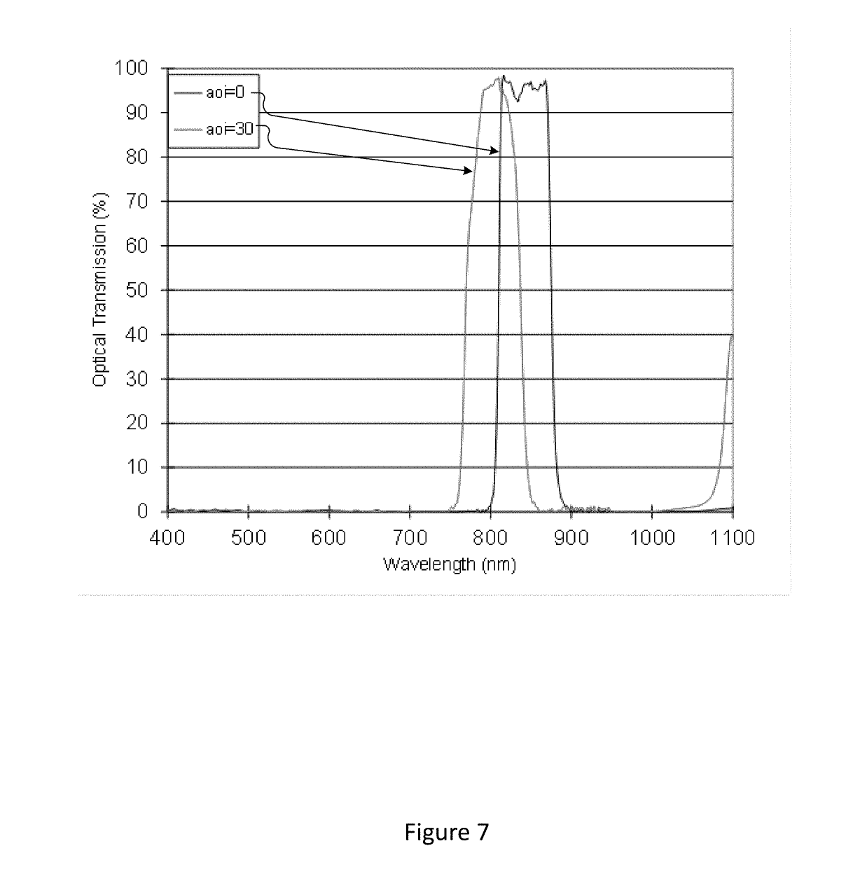

In some aspects, the filter 110 may be implemented as an optical filter. For example, the filter 110 may be a bandpass filter with spectral linewidth of 30 nm or less. Alternating layers of high and low index materials may be used to engineer the center wavelength and bandpass of the infrared bandpass filter (IRBF). Materials used may include one or more of TiO2, SiO2, Al2O3, and other thin film dielectric materials. In particular, the filter 110 may be an optical filter that includes a dielectric film that together with the remaining filter components provides a bandpass filter having a transfer function consistent with FIG. 7.

FIG. 7 illustrates a graph of the optical transmission of a filter according to an aspect of the disclosure. In particular, FIG. 7 illustrates a percent of optical transmission as shown in the vertical axis as compared to the wavelength in nanometers shown on the horizontal axis. Moreover, the graph shows two different optical transmissions for the filter 110 implemented as an optical filter. In particular, one line shows the percent optical transmission at an angle of incidence of 0°; and the other line shows the percent optical transmission at an angle of incidence of 30°. FIG. 7 shows that a filter operating consistent with the transfer function of FIG. 7 would pass frequencies generally in the 850 nm range and attenuate the remaining energy signals outside the passband. In this regard, the filter 110 may be implemented to enable a filter passband consistent with this transfer function.

In other aspects, the eye tracking system 100 may implement the filter 110 as an electrical filter that the filters electrical signals within the eye tracking system 100 to achieve a desired optical transmission and optical attenuation consistent with FIG. 7. In other aspects, the filter 110 may be implemented through digital signal processing in conjunction with the CPU 422 to achieve a desired optical transmission consistent with FIG. 7.

In a particular aspect, the IR light source 106 may utilize a VCSEL illumination source having a bandwidth less than 20 nm together with a narrow band IR Bandpass filter implementation of the filter 110 having a passband of approximately 20 nm. In a particular aspect, the IR light source 106 may utilize a VCSEL illumination source emitting infrared light centered on about 850 nm having a bandwidth less than 2.5 nm together with a narrow bandpass IR filter implementation of the filter 110 having a passband of approximately 20 nm centered on about 850 nm.

Additionally, ambient IR energy in the environment may also reach the filter 110. However, the filter 110 may filter out a substantial portion of that IR energy or ambient light except the portion 132 of IR energy reflected from the user's eye that reaches the IR sensor 107. This enables the IR camera 120 to capture only the image of the eye, without ambient light or with a reduced amount of ambient light.

Additionally, ambient IR energy in the environment may also reach the IR sensor 107. However, the fast shutter and TOF principles of the IR camera 120 enable the eye tracking system 100 to filter out all or filter a substantial portion of that IR energy except the portion 132 of IR energy reflected from the user's eye 199 that reaches the IR sensor 107. This can be done by setting the shutter 136 timing of the IR camera 120 so that IR energy transmitted from the eye tracking system 100 will be cut off by the shutter 136 (which may be electronic) on its way back to the IR sensor 107, so that only the portion 132 reflected to the IR sensor 107 (e.g., within a few centimeters) is captured by the IR camera 120; that is, only energy with a sufficiently predetermined short TOF is allowed to be captured. This enables the IR camera 120 to capture only, or substantially capture only, the image of the user's eye 199.

A further aspect of the eye tracking system 100 may include a timing adjustment device and/or algorithm. In this regard, certain aspects of the eye tracking system 100 rely on a discrete operation of the IR sensor 107 implemented together with discrete operation of the IR light source 106. In other words, the IR light source 106 is configured to provide short discrete pulses of light that are reflected off the user's eye 199 back to the IR sensor 107. The timing between operation of the IR light source 106 and the subsequent reception of light by the IR sensor 107 can vary depending on the distance between the IR sensor 107, the user's eye 199, and the IR light source 106. In particular, the location of each of these structures may be different for different implementations, may be different depending on a location of the various components, different for different components, and may be different based on the physiology of different users, and the like.

In this regard, the eye tracking system 100, the timing adjustment device, and/or algorithm may monitor the light pulses received by the IR sensor 107 and adjust the timing on a gating of the IR sensor 107 until the IR sensor 107 receives a maximum signal. For example, the timing on a gating of the IR sensor 107 may be varied from shortest reasonable capture time to longest reasonable capture time until the IR sensor 107 receives a maximum signal. The maximum signal strength being indicative of having the correct timing. Thereafter, this correct timing may be set and used for accurate control of time-of-flight operation of the IR light source 106, the IR sensor 107, the IR camera 120, and the shutter 136. Additionally, the process may be repeated from time to time to update the timing.

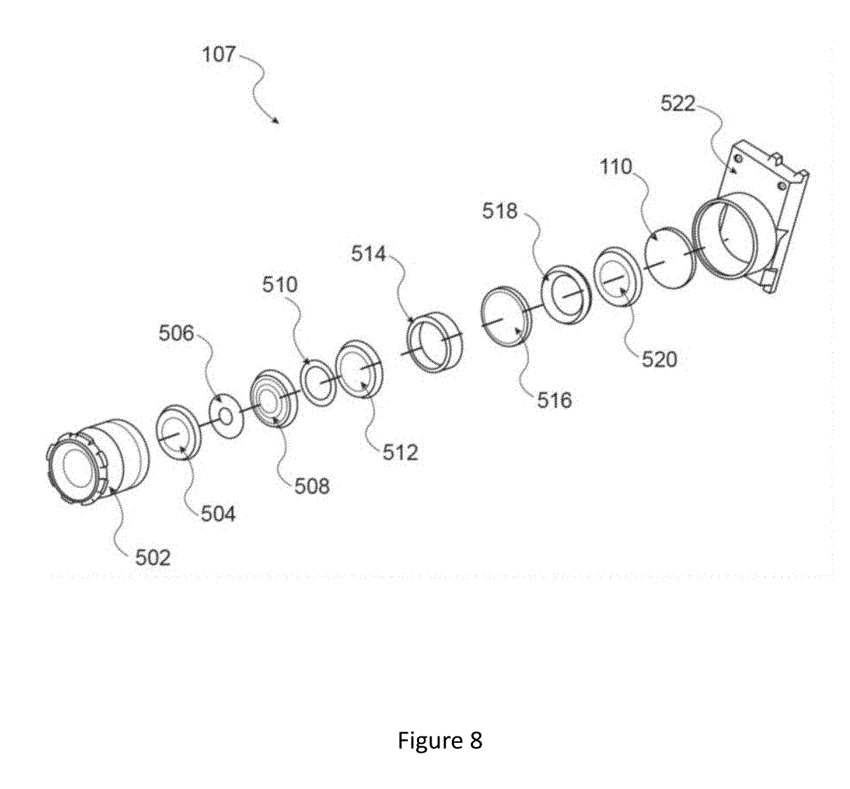

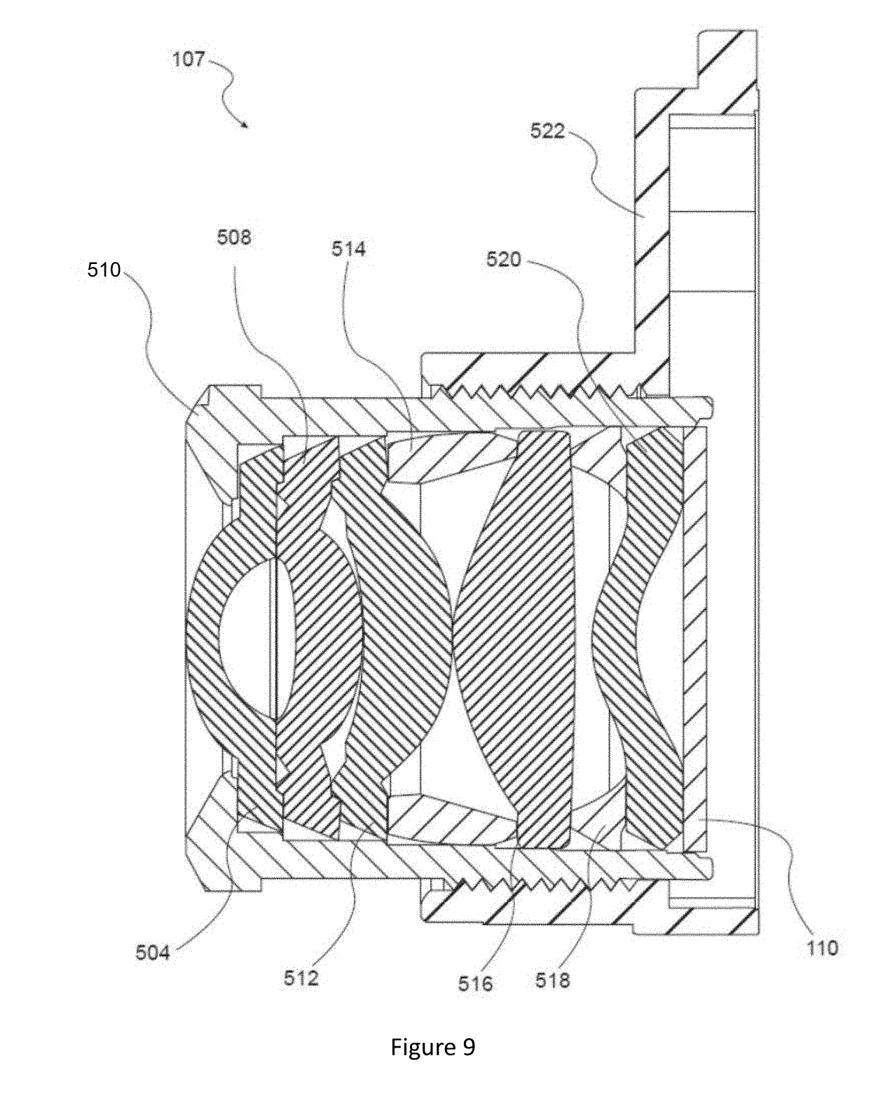

FIG. 8 illustrates an exploded view of a wide field of view (FOV) lens and the filter along with a holder according to an aspect of the disclosure; and FIG. 9 illustrates a cross-sectional view of the lenses and the filter along with the holder according FIG. 8. In particular, the IR sensor 107 may include a plurality of lenses, spacers, baffles, and the filter 110 all held by a barrel 502 and a holder 522. It should be noted that any combination including one or more of lenses, spacers, baffles, and the filter 110 is contemplated. In this regard, in some aspects fewer components may be utilized. In some aspects, components may be combined.

In a particular aspect, the barrel 502 may include a first lens 504, a second lens 508, a third lens 512, a fourth lens 516, and a fifth lens 520. In this particular aspect, the barrel 502 may further include a first baffle 506, a second baffle 510, a first spacer 514, and a second spacer 518. In some aspects, the barrel 502 may be fastened to the holder 522. In some aspects, the barrel 502 may be fastened to the holder 522 with the fastener. In some aspects, the barrel 502 may include a threaded male portion that is received by a threaded female portion of the holder 522.

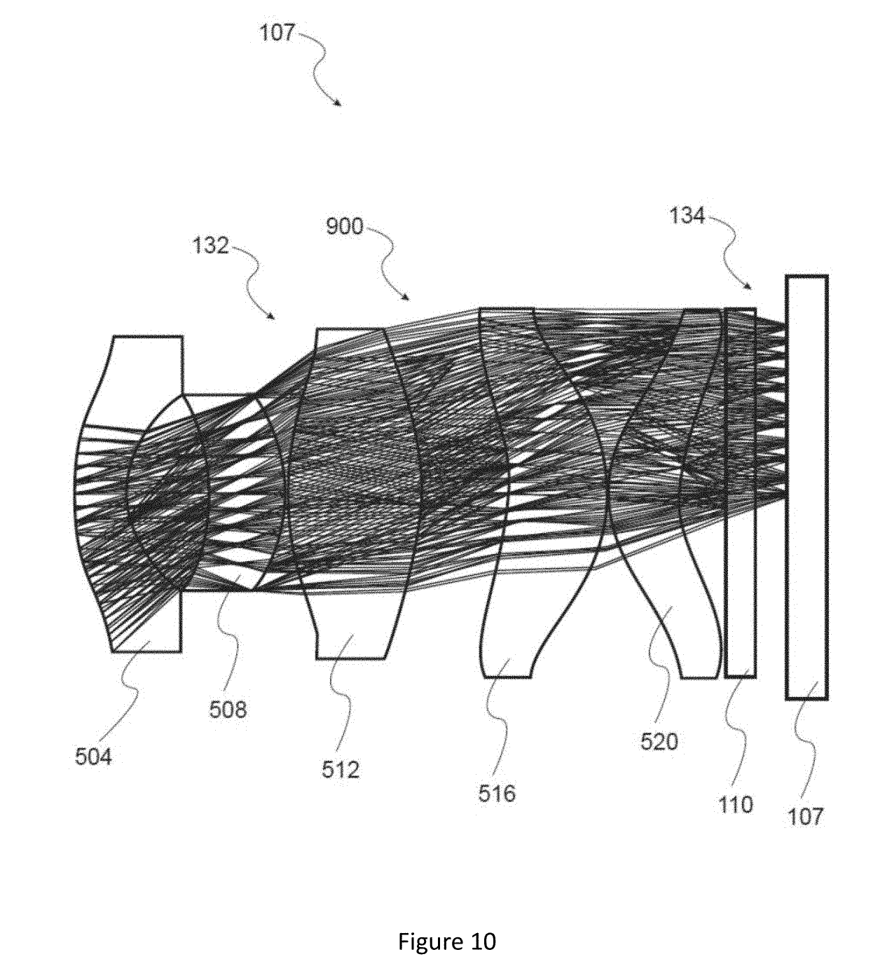

FIG. 10 illustrates a cross-sectional view of the lenses and the filter along with exemplary light transmission according to an aspect of the disclosure. In particular, FIG. 10 illustrates the transmission of light 900 through the first lens 504, the second lens 508, the third lens 512, the fourth lens 516, and the fifth lens 520. Moreover, FIG. 10 illustrates the transmission of the portion 132 through the filter 110. Finally, FIG. 10 illustrates a transfer of the filtered IR energy 134 to the IR sensor 107.

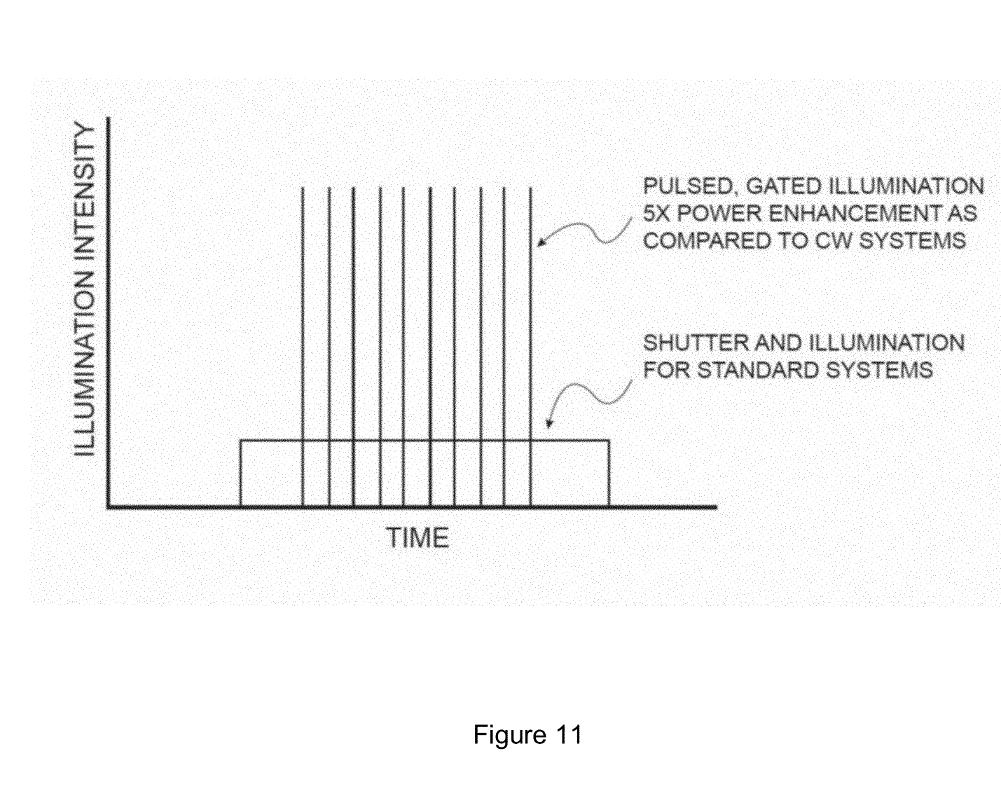

FIG. 11 graphically illustrates operation of the system using multiple short laser pulses in comparison to prior art systems. In this regard, FIG. 11 illustrates an illumination intensity along the vertical axis and time being along the horizontal axis. As further shown in FIG. 11, prior art solutions operated LEDs in quasi continuous wave (CW) mode, with no pulse power enhancement. Accordingly, the prior art illumination intensity is far less than the pulsed, gated illumination of the disclosure. In some aspects, the disclosed system may operate by using multiple short laser pulses and coordinating the shutter 136 with the light pulses as described above. In this regard, the total integration time can be reduced by the power enhancement achieved with laser light pulses. A typical value achievable using this technique is 2-10× the CW output power value. This corresponds to a 2 to 10× reduction in the total integration time, and a 2-10× reduction in the ambient light collection.

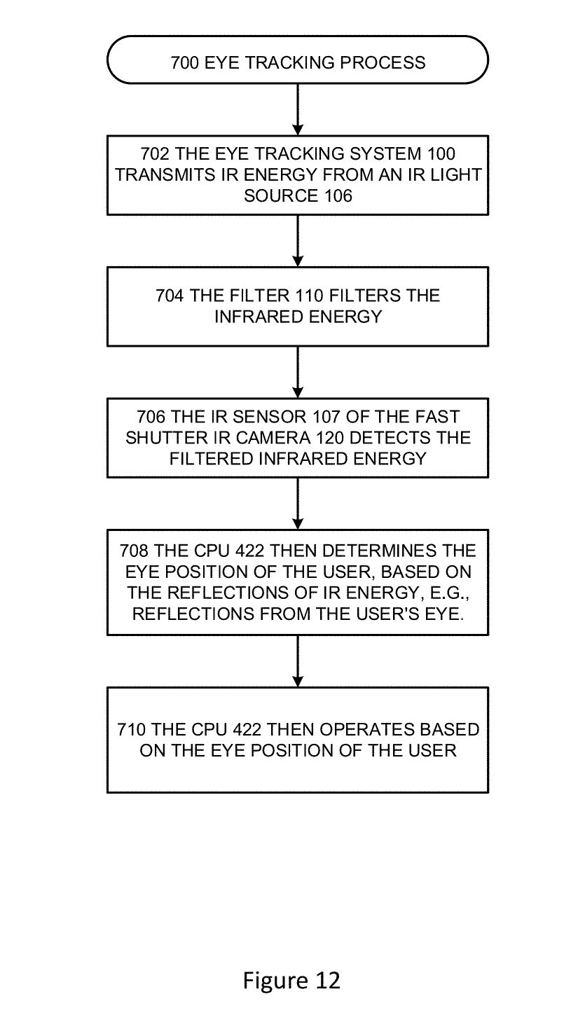

FIG. 12 is a flow diagram showing an example of the operational process of the eye tracking system according to some aspects. In particular, FIG. 12 illustrates an eye tracking process 700. At box 702 the eye tracking system 100 transmits IR energy 130 from an IR light source 106. A portion 132 of the transmitted IR energy 130 is reflected off the user's eye 199 back to the filter 110.

At box 704, the filter 110 filters the infrared energy to remove or substantially remove ambient light. The filter 110 filters the portion 132 and delivers a filtered IR energy 134 to the IR sensor 107 of the IR camera 120.

At box 706 the IR sensor 107 of the IR camera 120 detects filtered infrared energy. The IR camera 120 applies one or more gating functions at box 706 to filter out ambient IR from the detected IR, based on their TOFs. The filtered IR energy 134 that reaches the IR sensor 107 is detected and used by the CPU 422 (or alternatively by an external device) to determine the eye position (i.e., using pupil and/or iris identification), eye tracking, gesture recognition, object recognition, and the like.

The CPU 422 then determines at step 708 the eye position of the user, based on the IR energy, e.g., reflections from the user's eye. In box 710 the CPU 422 then utilizes the position for various applications. In one aspect, the CPU 422 modifies operation of the video display device 405 based on the eye position of the user.

Accordingly, the disclosure has set forth multiple techniques for mitigating the effects of ambient light in eye tracking systems used in a number of different applications. The disclosure has described a solution that isolates the returned light signal to specific distances. The advantages of this approach include at least: reduced sensitivity to ambient light; reduced overall illumination power; reduced sensitivity to parasitic reflections/stray light from other IR emitting sensors.

The disclosure has disclosed a time gated sensor with pulsed laser/LED to truncate light pulses to slices in depth from the camera source. By using coordinated fast light pulses and fast electronic shutter pulses to the sensor, the response has been shown to be limited to a specific distance that is defined as the time delay between the light pulse transmission and the sensor reception.

The disclosure has further described using multiple short laser pulses and coordinating the sensor shutter with the light pulses such that the total integration time can be reduced by the power enhancement achieved with laser light pulses. A typical value achievable using this technique is 2-5× the CW output power value.

### EXAMPLES OF CERTAIN ASPECTS

### Example 1

An eye movement tracking device comprising: an illumination source configured to transmit energy within a frequency band from a location proximate to an eye of a person such that a portion of transmitted energy is reflected off the eye of the person; a filter configured to filter a portion of the transmitted energy that is reflected off the eye of the person to generate filtered reflections; an image sensor and shutter configured to detect the filtered reflections of the portion of the transmitted energy, and to distinguish the filtered reflections of the portion of the transmitted energy from other energy detected by the image sensor and shutter based on times of flight and the frequency band of the filtered reflections of the portion of the transmitted energy and the other energy; and a processor configured to use the filtered reflections of the portion of the transmitted energy to determine a position of the eye of the person.

### Example 2

The eye movement tracking device according to Example 1, wherein the illumination source comprises a laser that is configured to generate and transmit infrared energy.

### Example 3

The eye movement tracking device according to one of Examples 1 to 2, wherein the laser comprises a Vertical Cavity Surface Emitting Laser (VCSEL).

### Example 4

The eye movement tracking device according to one of Examples 1 to 3, wherein the illumination source comprises one of the following: an LED or a laser; and wherein the illumination source is configured to have an output divergence less than 30°.

### Example 5

The eye movement tracking device according to one of Examples 1 to 4, wherein the illumination source comprises one of the following: a Vertical Cavity Surface Emitting Laser (VCSEL) or a laser; and wherein the illumination source is configured to have a spectral linewidth less than 10 nm.

### Example 6

The eye movement tracking device according to one of Examples 1 to 5, wherein the filter comprises a narrow bandpass filter having a passband less than 30 nm.

### Example 7

The eye movement tracking device according to one of Examples 1 to 6, wherein the processor is further configured to limit ambient energy in an environment based by utilizing the image sensor and shutter, a sensor shutter timing, and the filter to substantially filter out the ambient energy so that determination of a gaze direction of the eye is less affected by the ambient energy.

### Example 8

The eye movement tracking device according to one of Examples 1 to 7, wherein the illumination source is configured to generate and transmit infrared energy; wherein the illumination source comprises a Vertical Cavity Surface Emitting Laser (VCSEL); wherein the filter comprises a narrow bandpass filter; and wherein the narrow bandpass filter comprises an optical narrow bandpass filter.

### Example 9

he eye movement tracking device according to one of Examples 1 to 8, wherein the illumination source comprises one of the following: a Vertical Cavity Surface Emitting Laser (VCSEL) or a laser; wherein the illumination source is configured to have an output divergence less than 30°; and wherein the illumination source is configured to have a spectral linewidth less than 30 nm.

### Example 10

A process of tracking eye movement of a person, the method comprising: transmitting energy from an illumination source within a frequency band from a location proximate to an eye of the person such that a portion of the transmitted energy is reflected off the eye of the person; filtering the portion of the transmitted energy that is reflected off the eye of the person to generate filtered reflections with a filter; detecting the filtered reflections of the portion of the transmitted energy with an image sensor and shutter, and distinguishing the filtered reflections of the portion of the transmitted energy from other energy detected by the image sensor and shutter based on times of flight and said frequency band of the filtered reflections of the portion of the transmitted energy and the other energy; and determining a position of the eye of the person based on the filtered reflections of the portion of the transmitted energy with a processor.

### Example 11

The process of tracking eye movement of a person according to Example 10, wherein the illumination source comprises a laser that is configured to generate and transmit infrared energy.

### Example 12

The process of tracking eye movement of a person according to one of Examples 10 to 11, wherein the laser comprises a Vertical Cavity Surface Emitting Laser (VCSEL).

### Example 13

The process of tracking eye movement of a person according to one of Examples 10 to 12, wherein the illumination source comprises one of the following: an LED or a laser; and wherein the illumination source is configured to have an output divergence less than 30°.

### Example 14

The process of tracking eye movement of a person according to one of Examples 10 to 13, wherein the illumination source comprises one of the following: a Vertical Cavity Surface Emitting Laser (VCSEL) or a laser; and wherein the illumination source is configured to have a spectral linewidth less than 30 nm.

### Example 15

The process of tracking eye movement of a person according to one of Examples 10 to 14, wherein the filter comprises a narrow bandpass filter.

### Example 16

The process of tracking eye movement of a person according to one of Examples 10 to 15, wherein the processor is further configured to limit ambient energy in an environment by utilizing the image sensor and shutter, a shutter window, and the filter to substantially filter out the ambient energy so that determination of a gaze direction of the eye is less affected by the ambient energy.

### Example 17

The process of tracking eye movement of a person according to one of Examples 10 to 16, wherein the illumination source is configured to generate and transmit infrared energy; wherein the illumination source comprises one of the following: a Vertical Cavity Surface Emitting Laser (VCSEL) or a laser; wherein the filter comprises a narrow bandpass filter; and wherein the narrow bandpass filter comprises an optical narrow bandpass filter.

### Example 18

The process of tracking eye movement of a person according to one of Examples 10 to 17, wherein the illumination source is configured to have an output divergence less than 30°; and wherein the illumination source is configured to have a spectral linewidth less than 10 nm.

### Example 19

An eye movement tracking device comprising: means for transmitting energy within a frequency band from a location proximate to an eye of a person such that a portion of the transmitted energy is reflected off the eye of the person; means for filtering the portion of the transmitted energy that is reflected off the eye of the person to generate filtered reflections; means for detecting the filtered reflections of the portion of the transmitted energy, and means for distinguishing the filtered reflections of the portion of the transmitted energy from other energy detected based on times of flight and said frequency band of the filtered reflections of the portion of the transmitted energy and the other energy; and means for determining a position of the eye of the person based on the filtered reflections of the portion of the transmitted energy.

### Example 20

The eye movement tracking device according to Example 19, wherein the means for transmitting energy is configured to generate and transmit infrared energy; wherein the means for transmitting energy comprises one of the following: a Vertical Cavity Surface Emitting Laser (VCSEL) or a laser; and wherein the means for filtering comprises a narrow bandpass filter; and wherein the narrow bandpass filter comprises an optical narrow bandpass filter.

The machine-implemented operations described above can be implemented by programmable circuitry programmed/configured by software and/or firmware, or entirely by special-purpose circuitry, or by a combination of such forms. Such special-purpose circuitry (if any) can be in the form of, for example, one or more application-specific integrated circuits (ASICs), programmable logic devices (PLDs), field-programmable gate arrays (FPGAs), system-on-a-chip systems (SOCs), etc.

Software or firmware to implement the techniques introduced here may be stored on a non-transitory machine-readable storage medium and may be executed by one or more general-purpose or special-purpose programmable microprocessors. A “machine-readable medium,” as the term is used herein, includes any mechanism that can store information in a form accessible by a machine (a machine may be, for example, a computer, network device, cellular phone, personal digital assistant (PDA), manufacturing tool, any device with one or more processors, etc.). For example, a machine-accessible medium includes recordable/non-recordable media (e.g., read-only memory (ROM); random access memory (RAM); magnetic disk storage media; optical storage media; flash memory devices; etc.), etc. Machine-readable storage media do not include signals.

Aspects of the disclosure may include communication channels that may be any type of wired or wireless electronic communications network, such as, e.g., a wired/wireless local area network (LAN), a wired/wireless personal area network (PAN), a wired/wireless home area network (HAN), a wired/wireless wide area network (WAN), a campus network, a metropolitan network, an enterprise private network, a virtual private network (VPN), an internetwork, a backbone network (BBN), a global area network (GAN), the Internet, an intranet, an extranet, an overlay network, Near field communication (NFC), a cellular telephone network, a Personal Communications Service (PCS), using known protocols such as the Global System for Mobile Communications (GSM), CDMA (Code-Division Multiple Access), GSM/EDGE and UMTS/HSPA network technologies, Long Term Evolution (LTE), 5G (5th generation mobile networks or 5th generation wireless systems), WiMAX, HSPA+, W-CDMA (Wideband Code-Division Multiple Access), CDMA2000 (also known as C2K or IMT Multi-Carrier (IMT-MC)), Wireless Fidelity (Wi-Fi), Bluetooth, and/or the like, and/or a combination of two or more thereof. The NFC standards cover communications protocols and data exchange formats, and are based on existing radio-frequency identification (RFID) standards including ISO/IEC 14443 and FeliCa. The standards include ISO/IEC 18092[3] and those defined by the NFC Forum.

Aspects of the disclosure may be implemented in any type of computing devices, such as, e.g., a desktop computer, personal computer, a laptop/mobile computer, a personal data assistant (PDA), a mobile phone, a tablet computer, cloud computing device, and the like, with wired/wireless communications capabilities via the communication channels.

Aspects of the disclosure may be implemented in any type of mobile smartphones that are operated by any type of advanced mobile data processing and communication operating system, such as, e.g., an Apple™ iOS™ operating system, a Google™ Android™ operating system, a RIM™ Blackberry™ operating system, a Nokia™ Symbian™ operating system, a Microsoft™ Windows Mobile™ operating system, a Microsoft™ Windows Phone™ operating system, a Linux™ operating system or the like.

Further in accordance with various aspects of the disclosure, the methods described herein are intended for operation with dedicated hardware implementations including, but not limited to, PCs, PDAs, semiconductors, application specific integrated circuits (ASIC), programmable logic arrays, cloud computing devices, and other hardware devices constructed to implement the methods described herein.

It should also be noted that the software implementations of the disclosure as described herein are optionally stored on a non-transitory tangible storage medium, such as: a magnetic medium such as a disk or tape; a magneto-optical or optical medium such as a disk; or a solid state medium such as a memory card or other package that houses one or more read-only (non-volatile) memories, random access memories, or other re-writable (volatile) memories. A digital file attachment to email or other self-contained information archive or set of archives is considered a distribution medium equivalent to a tangible storage medium. Accordingly, the disclosure is considered to include a non-transitory tangible storage medium or distribution medium, as listed herein and including art-recognized equivalents and successor media, in which the software implementations herein are stored.

Additionally, the various aspects of the disclosure may be implemented in a non-generic computer implementation. Moreover, the various aspects of the disclosure set forth herein improve the functioning of the system as is apparent from the disclosure hereof. Furthermore, the various aspects of the disclosure involve computer hardware that it specifically programmed to solve the complex problem addressed by the disclosure. Accordingly, the various aspects of the disclosure improve the functioning of the system overall in its specific implementation to perform the process set forth by the disclosure and as defined by the claims.

While the disclosure has been described in terms of exemplary aspects, those skilled in the art will recognize that the disclosure can be practiced with modifications in the spirit and scope of the appended claims. These examples given above are merely illustrative and are not meant to be an exhaustive list of all possible designs, aspects, applications or modifications of the disclosure.

What is claimed is:

1. An eye movement tracking device comprising:
an illumination source configured to transmit energy within a frequency band from a location proximate to an eye of a person such that a portion of transmitted energy is reflected off the eye of the person;
a filter configured to filter a portion of the transmitted energy that is reflected off the eye of the person to generate filtered reflections;
an image sensor and shutter configured to detect the filtered reflections of the portion of the transmitted energy, and to distinguish the filtered reflections of the portion of the transmitted energy from other energy detected by the image sensor and shutter based on times of flight and the frequency band of the filtered reflections of the portion of the transmitted energy and the other energy; and
a processor configured to use the filtered reflections of the portion of the transmitted energy to determine a position of the eye of the person.

2. The eye movement tracking device according to claim 1, wherein the illumination source comprises a laser that is configured to generate and transmit infrared energy.

3. The eye movement tracking device according to claim 2, wherein the laser comprises a Vertical Cavity Surface Emitting Laser (VCSEL).

4. The eye movement tracking device according to claim 1, wherein the illumination source comprises one of the following: an LED or a laser; and wherein the illumination source is configured to have an output divergence less than 30°.

5. The eye movement tracking device according to claim 1, wherein the illumination source comprises one of the following: a Vertical Cavity Surface Emitting Laser (VCSEL) or a laser; and wherein the illumination source is configured to have a spectral linewidth less than 10 nm.

6. The eye movement tracking device according to claim 1, wherein the filter comprises a narrow bandpass filter having a passband less than 30 nm.

7. The eye movement tracking device according to claim 1, wherein the processor is further configured to limit ambient energy in an environment based by utilizing the image sensor and shutter, a sensor shutter timing, and the filter to substantially filter out the ambient energy so that determination of a gaze direction of the eye is less affected by the ambient energy.

8. The eye movement tracking device according to claim 1, wherein the illumination source is configured to generate and transmit infrared energy; wherein the illumination source comprises a Vertical Cavity Surface Emitting Laser (VCSEL); wherein the filter comprises a narrow bandpass filter; and wherein the narrow bandpass filter comprises an optical narrow bandpass filter.

9. The eye movement tracking device according to claim 1, wherein the illumination source comprises one of the following: a Vertical Cavity Surface Emitting Laser (VCSEL) or a laser; wherein the illumination source is configured to have an output divergence less than 30°, and wherein the illumination source is configured to have a spectral linewidth less than 30 nm.

10. A process of tracking eye movement of a person, the method comprising:
transmitting energy from an illumination source within a frequency band from a location proximate to an eye of the person such that a portion of the transmitted energy is reflected off the eye of the person;
filtering the portion of the transmitted energy that is reflected off the eye of the person to generate filtered reflections with a filter;
detecting the filtered reflections of the portion of the transmitted energy with an image sensor and shutter, and distinguishing the filtered reflections of the portion of the transmitted energy from other energy detected by the image sensor and shutter based on times of flight and said frequency band of the filtered reflections of the portion of the transmitted energy and the other energy; and
determining a position of the eye of the person based on the filtered reflections of the portion of the transmitted energy with a processor.

11. The process of tracking eye movement of a person according to claim 10, wherein the illumination source comprises a laser that is configured to generate and transmit infrared energy.

12. The process of tracking eye movement of a person according to claim 11, wherein the laser comprises a Vertical Cavity Surface Emitting Laser (VCSEL).

13. The process of tracking eye movement of a person according to claim 10, wherein the illumination source comprises one of the following: an LED or a laser; and wherein the illumination source is configured to have an output divergence less than 30°.

14. The process of tracking eye movement of a person according to claim 10, wherein the illumination source comprises one of the following: a Vertical Cavity Surface Emitting Laser (VCSEL) or a laser; and wherein the illumination source is configured to have a spectral linewidth less than 30 nm.

15. The process of tracking eye movement of a person according to claim 10, wherein the filter comprises a narrow bandpass filter.

16. The process of tracking eye movement of a person according to claim 10, wherein the processor is further configured to limit ambient energy in an environment by utilizing the image sensor and shutter, a shutter window, and the filter to substantially filter out the ambient energy so that determination of a gaze direction of the eye is less affected by the ambient energy.

17. The process of tracking eye movement of a person according to claim 10, wherein the illumination source is configured to generate and transmit infrared energy; wherein the illumination source comprises one of the following: a Vertical Cavity Surface Emitting Laser (VCSEL) or a laser; wherein the filter comprises a narrow bandpass filter; and wherein the narrow bandpass filter comprises an optical narrow bandpass filter.

18. The process of tracking eye movement of a person according to claim 17, wherein the illumination source is configured to have an output divergence less than 30°, and wherein the illumination source is configured to have a spectral linewidth less than 10 nm.

19. An eye movement tracking device comprising:
means for transmitting energy within a frequency band from a location proximate to an eye of a person such that a portion of the transmitted energy is reflected off the eye of the person;
means for filtering the portion of the transmitted energy that is reflected off the eye of the person to generate filtered reflections;
means for detecting the filtered reflections of the portion of the transmitted energy, and means for distinguishing the filtered reflections of the portion of the transmitted energy from other energy detected based on times of flight and said frequency band of the filtered reflections of the portion of the transmitted energy and the other energy; and
means for determining a position of the eye of the person based on the filtered reflections of the portion of the transmitted energy.

20. The eye movement tracking device according to claim 19, wherein the means for transmitting energy is configured to generate and transmit infrared energy; wherein the means for transmitting energy comprises one of the following: a Vertical Cavity Surface Emitting Laser (VCSEL) or a laser; and wherein the means for filtering comprises a narrow bandpass filter; and wherein the narrow bandpass filter comprises an optical narrow bandpass filter.
